(19) United States
(12) Patent Application Publication
(10) Pub. No.: US 2007/0134373 A1
(43) Pub. Date: Jun. 14, 2007

(54) Process for the preparation of galactose

(76) Inventors: Giovanni Cipoletti, Milano (IT); Marco Manoni, Milano (IT); Luana Vagnoli, Arezzo (IT); Silvia Giacomelli, Sesto Fiorentino (IT); Silvia Biagiolini, Arezzo (IT); Cristina Pratesi, Piteglio (IT); Lucia Camici, Monsummano Terme (IT)

Correspondence Address: Jay S. Cinamon;Abelman, Frayne and Schwab, 666 Third Avenue, New York, NY, 10017-5621, US

(21) Appl. No.: 10/577,847

(22) PCT Filed: Oct. 28, 2004
(86) PCT No.: PCT/EP04/52709

§ 371 Date: Apr. 27, 2006

(30) Foreign Application Priority Data
Oct. 29, 2003 (IT) FI2003A000275

(51) Int. Cl. A23C 9/12; C12P 19/02

(52) U.S. Cl. 426/34; 435/105

(57) ABSTRACT

A process for the preparation of high purity galactose is described, comprising the inoculum, of milk or milk serum not previously subjected to any type of preliminary treatment and/or purification procedure and not containing any bactericides or bacteriostats, with not modified micro-organisms commonly used in dairy industry.

### FIELD OF INVENTION

The present invention concerns the field of processes for the production of simple sugars, and in particular a process for the preparation of galactose.

### PRIOR ART

Galactose is one of the two simple sugars that constitutes the lactose molecule; it is used both in the food industry as sweetener and as intermediate in many chemical syntheses.

The production of galactose starting from the lactose contained in milk and in milk derivatives, both by means of enzymatic and chemical hydrolysis, is known.

In order to obtain the final product with good yields, in both of these processes it is necessary to make a preliminary removal of the protein portion of milk, for example through one or more ultra filtration steps as described in European Patent Application No. 168 127, or by warm acid precipitation of proteins.

Also in the U.S. Pat. No. 3,981,773, that describes a process for the preparation of galactose by inoculum of a solution containing lactose with specific yeasts or bacteria, the necessity to remove proteins before fermentation is highlighted. Moreover, according to the above said patent, the fermentation cannot be carried out with any kind of micro-organism, but a few modified yeasts or bacteria have to be purpose-selected. Still according to U.S. Pat. No. 3,981,773 if such modified bacteria are used, it is necessary to extract galactose from fermentation residues by concentration of the so obtained galactose solution, by one or more crystallisation steps, preceded by a step on active coal or an extraction with ethanol.

Moreover, both chemical and enzymatic hydrolyses require the removal of glucose by using various kind of micro-organisms, such as yeasts belonging to Saccaromyces genus, or by operating enzymatically still with the glucose-oxidase enzyme able to transform glucose into gluconic acid.

The main limits of the above described known processes are of economical nature: the need to use enzymes in one or more steps and the necessity to purify the starting material increase greatly the production costs. The process carried out directly on milk serum appears to be, in fact, very difficult both in chemical and enzymatic way. On one hand, the chemical way requires the use of strong acids at high temperatures, which causes the formation of carbonaceous coloured substances that derive from thermal degradation of the organic substance; on the other hand, in the enzymatic way, the presence of materials of varied nature in suspension, decreases the enzyme efficiency.

Therefore, the need of a process for preparing galactose not having the disadvantages above described for the known processes and allowing to prepare galactose with high purity, directly employable in food industry and in chemical syntheses without being necessarily subjected to complex purification processes, is deeply felt.

### SUMMARY OF THE INVENTION

The Applicant has now surprisingly found that galactose with high purity can be obtained by inoculating with non modified micro-organisms commonly used in dairy industry, milk or milk serum not subjected to any preliminary and purification treatment and not containing bactericides or bacteriostats.

It is therefore subject of the invention a process for the preparation of galactose starting from milk or milk serum not subjected to any preliminary and purification treatment and not containing any bactericides or bacteriostats, comprising the following step:
i) inoculum of milk or milk serum with non modified micro-organisms able to hydrolyse lactose thus obtaining galactose and glucose, and to consume the so obtained glucose;
ii) fermentation of the solution coming from step i);
iii) recovery of the desired galactose solution from the fermentation product coming from step ii).

A further subject of the invention is a method for disposal of milk serum derived from dairy industry containing at least 2.5% by weight of lactose in respect to the total weight not subjected to any preliminary and purification treatment and not containing bactericides or bacteriostats, comprising inoculating serum with non modified micro-organisms able to hydrolyse lactose thus obtaining galactose and glucose and to consume the so obtained glucose, followed by fermentation and recovery of a galactose solution from the fermentation product according to the present process as described above.

Features and advantages of the present invention will be illustrated in details in the following description.

### DETAILED DESCRIPTION OF THE INVENTION

The process according to the invention can be successfully used with any milk serum, milk or sprayed serum in powder reconstituted with water not previously subjected to any preliminary and purification treatment, with the proviso that it does not contain any bactericides or bacteriostats.

If not otherwise indicated, by the expression “milk serum” according to the present invention any milk serum not containing bactericides or bacteriostats is meant, also sprayed milk serum in powder reconstituted with water or milk serum impoverished in lactose or milk proteins as directly arriving from dairy industry.

The concentration of lactose in the starting milk or milk serum according to the is invention preferably ranges between 2.5% by weight with respect to the total weight of the milk or milk serum and the saturation concentration; optimal results are obtained when the lactose is in amount ranging between 3 and 15% by weight.

According to a particular embodiment of the invention the starting milk or milk serum, if necessary, is brought to pH≦7.5, and preferably to a pH ranging between 5.0 and 7.5, by adding a base, weak or strong, preferably of inorganic origin, chosen for example in the group consisting of sodium hydroxide, potassium hydroxide, calcium hydroxide, magnesium oxide, calcium carbonate and ammonia; the so obtained suspension is then pasteurised to remove possible microbial charges antagonistic to that of the ferment for the inoculum in step i) of the process.

After the possible pasteurisation, the temperature of the solution is left cooling down to a temperature typically ranging between 25 and 50° C. This is the temperature at which the inoculum and fermentation steps are carried out; preferably the temperature is left cooling down until a value comprised in the range between 37 and 45° C.

Any non modified micro-organisms able to hydrolyse lactose thus obtaining galactose and glucose and to consume the so obtained glucose, can be efficaciously used in this process. Of possible use are for example the natural micro-organisms used in dairy industry and generally named “lactic ferments” or “yoghurt ferments”.

Moreover, any composition comprising such micro-organisms has to be considered as included in the scope of the present invention.

According to a particular embodiment of the invention, the process comprises the fermentation of the milk or milk serum containing lactose with bacteria belonging to the family of Lactobacillaceae or with compositions thereof.

According to the invention “family of Lactobacillaceae” means the family so named according to the classification reported in Bergey's Manual of Determinative Bacteriology, 7^{th} Ed., 1957. Among the bacteria belonging to the Lactobacillaceae family, the bacteria belonging to Streptococcus and Lactobacillus genus and their mixture, are preferred for the inoculum of milk or milk serum in step i) of the present process. Examples of these bacteria are the bacteria belonging to the bacterial stocks selected from the group consisting of Streptococcus Thermophilus, Lactobacillus Bulgaricus, Lactobacillus Casei and mixtures thereof.

The Applicant has verified that in order to obtain the greater glucose consumption coming from the lactose hydrolysis, avoiding that great quantities of galactose are consumed by bacteria, the fermentation step ii) is preferably carried out maintaining a constant pH at a value ≦7.5, and more preferably at a pH value ranging between 5.0 and 7.5, for a period of time between 16 and 24 hours. If necessary, to further decrease the lactose concentration keeping practically unchanged the quantity of the so formed galactose, this first fermentation step at a constant pH is followed by a second step in which pH is left spontaneously decreasing by formation of lactic acid due to the further fermentation, for a period of time between 5 and 60 hours.

During fermentation, the suspension is preferably kept under constant stirring. In the fermentation step at constant pH, the pH value can be kept in the preferred range by adding a base, weak or strong, preferably inorganic, selected for example amongst the above mentioned inorganic bases.

At the end of fermentation step, before carrying out step iii), a second pasteurisation can be possibly carried out as the previous pasteurisation, according to the commonly used procedures known to any skilled person.

The recovery of the desired galactose solution from the product of fermentation step ii) is carried out removing by centrifugation and/or ultra filtration the biomass, consisting of fat, denatured proteins not derived from serum or from bacterial cells coming from fermentation.

The product coming from ultrafiltration may be possibly subjected to nanofiltration to remove the lactose if present in non negligible concentration. This may occur for example when the starting serum contains lactose in concentration of 16% by weight or higher.

According to a particular embodiment of this process, after having removed the biomass, the so obtained solution, clear and yellow, is deionised by electrodyalisis until a conductivity of 6-0.5 ms and subsequent passage through an ion exchange column consisting in a strong cationic resin in form H⁺ and a weak anionic resin in form OH⁻, where the conductivity is further decreased to 10-100 μs. The so obtained solution can be then micro-filtered for example with a membrane 0.1-0.8 μm.

After removal of salts from the so obtained solution, the water can be removed (for instance through inverse osmosis or through distillation at reduced pressure) thus obtaining a syrup with the desired galactose concentration, or the galactose can be crystallised, from sufficiently concentrated solutions, according to the common procedures known to any person skilled in the art.

The galactose solutions obtained with the present process have a galactose content of around 90% by weight with respect to the total weight of the dry substance that is present in solution, a content of lactose lower than 10% and a negligible quantity of galactosyl-galactosides. Therefore, these solutions can be directly used as sweetener in galactose drinks and in other food preparations; they can be used as well to obtain pure galactose with excellent crystallisation yields, to be used, for example, as synthesis intermediate in various chemical processes.

Besides that, advantageously the present process does not require the preliminary removal of the proteic portion in order to have better yields of the final product, nor requires the removal of glucose that is directly consumed by the micro-organisms used in fermentation.

The process of the invention can be used successfully with serum directly derived from dairy industry and not previously subjected to any purification process.

This latest advantageous aspect of the present process allows its application also in a method for disposal of the milk serum, a pollutant difficult to be removed for dairy industries.

The following examples are reported as a non-limiting illustration of the invention.

### EXAMPLE 1

3 litres of fresh milk serum containing 3.5% of lactose are pasteurised at 90° C., then thermostated at 37° C. and brought to pH=7 with a 30% aqueous solution of NaOH.

Inoculation is carried out with stocks of Streptococcus Thermophilus, Lactobacillus Bulgaricus and Lactobacillus Casei in mixture. After 18 hours under stirring, during which the pH value is maintained at pH=7 with NaOH 30%, the supply of the base is interrupted and the mixture is left under spontaneous acidification. After further 8 hours the amount of lactose is 0.13% by weight and the amount of galactose is 1.34% by weight.

After pasteurisation at 90° C., the biomass is removed by centrifugation and subsequent ultrafiltration. The solution coming from ultrafiltration is then demineralised by electrodyalisis and passage on ion exchange column.

The solution can possibly be decolourised on carbon, microfiltrated and finally concentrated to obtain a syrup.

### EXAMPLE 2

The starting material is sprayed milk serum in powder; it is reconstituted with demineralised H₂O so to obtain 2000 g of a suspension containing an amount of lactose of 5.5% by weight in respect of the total weight.

The so obtained fermentation substrate is pasteurised at 90° C., then thermostated at 40° C. and brought to pH=6.5 with an aqueous solution of NH₃ having a concentration of 7.5% by weight.

Inoculation is carried out with stocks of Streptococcus Thermophilus maintaining the mixture under slow stirring and the pH value at pH=6.5 still with NH₃ 7.5%.

After 21 hours the amount of lactose is 0.28% by weight and the amount of galactose is 2.30% by weight.

The culture broth is pasteurised again at 90° C. and the biomass is removed by centrifugation and subsequent ultrafiltration. The centrifugated is subjected to electrodyalisis. Demineralisation is then completed on ion exchange resins (strong cationic and weak anionic resins).

After decolourisation the water is evaporated until a concentration of 65° Brix is obtained to crystallise galactose according to known methods: crystalline galactose having a purity of 99% is obtained, in an amount of 31 g, corresponding to 82% of the sugar present in solution before concentration.

### EXAMPLE 3

4.5 litres of fresh serum containing 3.4% by weight of lactose are pasteurised at 80° C., then thermostated at 45° C.

The pH is brought to pH=6.8 with an aqueous solution of KOH having a concentration of 40%, and the inoculum is accomplished with the commercial product sold under the trade name Actimel® and containing yoghurt ferments.

The pH value is maintained at pH=6.8 with KOH 40% for 24 hours, afterwards a pasteurisation at 80° C. is carried out, thus obtaining a fermentation product containing 0.04% by weight of lactose and 1.04% by weight of galactose. Then the biomass is removed by centrifugation and subsequent ultrafiltration. The ultrafiltrated is then demineralised by passage on ion exchange column.

The solution can possibly be decolourised on carbon, microfiltrated and finally concentrated into syrup.

### EXAMPLE 4

Milk serum powder is reconstituted with demineralised H₂O obtaining 10,700 kg of a suspension containing 7.34% by weight of lactose. The so obtained fermentation substrate is pasteurised at 80° C., thermostated at 40° C. and brought to pH=7 with an aqueous solution of NaOH having a concentration of 30%.

The inoculum is carried out with a mixture of stocks of Streptococcus Thermophilus and Lactobacillus Bulgaricus. After 18 hours under slow stirring and maintaining the pH value at pH=7 by adding NaOH 30%, the supply of NaOH is interrupted and the pH value spontaneously decreases.

After 10 hours in acidification, a pasteurisation at 80° C. is carried out: the lactose is 0.23% by weight and the galactose is 2.95% by weight

The biomass is removed by ultrafiltration; on the ultrafiltrated the complete demineralisation is carried out on ion exchange resins (strong cationic and weak anionic resins).

From this solution, after a possible decolourisation, the water is removed until a syrup is obtained, having the desired concentration and able to be microfiltered. The galactose in this syrup has a purity of 89%.

### EXAMPLE 5

28 Kg of milk serum powder are reconstituted with demineralised H₂O so as to obtain a total volume of 110 litres. The content of lactose in the so obtained suspension is 16.26% by weight

This fermentation substrate is pasteurised at 90° C. for 1 min, thermostated at 40° C. and brought to pH=6.5 with an aqueous suspension of Ca(OH)₂ having a concentration of 15% w/v.

The inoculum is carried out with 5 g of Streptococcus Thermophilus.

After 40 hours under slow stirring and maintaining the pH value at pH=6.4-6.5 by adding Ca(OH)₂ 15% w/v, a pasteurisation at 80° C. is carried out, thus obtaining a fermentation product containing 1.76% by weight of lactose and 3.88% by weight of galactose. Then the biomass is removed by centrifugation and subsequent ultrafiltration. The ultrafiltrated is then nanofiltrated to remove lactose, and demineralised by passage on ion exchange column.

The solution is microfiltrated and finally concentrated into a syrup of galactose having a purity of 90%.

1. A process for the preparation of galactose starting from milk or milk serum not subjected to any preliminary and purification treatment and not containing any bactericides or bacteriostats, comprising the following step:
i) inoculum of milk or milk serum with non modified micro-organisms able to hydrolyse lactose thus obtaining galactose and glucose, and to consume the so obtained glucose;
ii) fermentation of the solution coming from step i) maintaining a constant pH value at pH≦7.5 for a period of time ranging between 16 and 24 hours, by adding a base, strong or weak, of inorganic origin;
iii) recovery of the desired galactose solution from the fermentation product coming from step ii).

2. The process according to claim 1, in which said milk or milk serum has a concentration in lactose ranging between 2.5% by weight in respect to the total weight of the milk or milk serum and the saturation concentration.

3. The process according to claim 2, in which said milk or milk serum has a concentration in lactose ranging between 3 and 15% by weight in respect to the total weight of the milk or milk serum.

4. The process according to claim 1, in which said non modified micro-organisms in step i) are selected from between lactic ferments and yoghurt ferments.

5. The process according to claim 1, in which said non modified micro-organisms of step i) are selected from among bacteria belonging to the family of Lactobacillaceae.

6. The process according to claim 5, in which said bacteria belonging to the family of Lactobacillaceae are bacteria belonging to bacterial stocks selected from the group consisting of Streptococcus, Lactobacillus and mixtures thereof.

7. The process according to claim 6, in which said bacteria are selected from the group consisting of Streptococcus Thermophilus, Lactobacillus Bulgaricus, Lactobacillus Casei species and mixtures thereof.

8. The process according to claim 1, in which in said fermentation in step ii) after the period of time in which the pH value is maintained constant at pH≦7.5, the pH value is then allowed to spontaneously decrease for a period of time ranging between 5 and 60 hours.

9. The process according to claim 1, in which said constant pH value ranges between 5.0 and 7.5.

10. The process according to claim 1, in which said fermentation in step ii) is carried out at a temperature ranging between 25 and 50° C.

11. The process according to claim 10, in which said fermentation in step ii) is carried out at a temperature ranging between 37 and 45° C.

12. The process according to claim 1, in which said milk or milk serum, before being subjected to inoculum in step i), if necessary, is brought to a pH≦7.5.

13. The process according to claim 12, in which said milk or milk serum, before being subjected to inoculum in step i), is brought to a pH ranging between 5.0 and 7.5.

14. The process according to claim 12, in which said pH≦7.5 value is obtained by adding a base, strong or weak, of inorganic origin.

15. The process according to claims 1, in which said base of inorganic origin added in step ii) is selected from the group consisting of sodium hydroxide, potassium hydroxide, calcium hydroxide, magnesium hydroxide, calcium carbonate and ammonia.

16. The process according to claim 1, in which the recovery of the galactose solution from the product of fermentation in step ii) is carried out removing the biomass by centrifugation and/or ultrafiltration, thus obtaining a solution that is possibly nanofiltrated and/or concentrated at warm under vacuum, to remove water and obtain a galactose solution of the desired concentration.

17. The process according to claim 16, in which after removal of the biomass, the resulting solution is deionised by electrodyalisis and subsequent passage on an ion exchange column, and microfiltrated.

18. The process according to claim 1, in which said milk or milk serum, before being subjected to inoculum in step i), and/or at the end of fermentation in step ii), is subjected to pasteurisation.

19. Method for the disposal of milk serum derived from dairy industry containing at least 2.5% by weight of lactose in respect to the total weight not subjected to any preliminary and purification treatment and not containing bactericides or bacteriostats, comprising inoculating serum with non modified micro-organisms able to hydrolyse lactose thus obtaining galactose and glucose and to consume the so obtained glucose, followed by fermentation maintaining a constant pH value at pH≦7.5 for a period of time ranging between 16 and 24 hours, by adding a base, strong or weak, of inorganic origin, and finally recovery of a galactose solution from the fermentation product.

20. The process according to claim 14, in which said base, strong or weak, of inorganic origin is selected from the group consisting of sodium hydroxide, potassium hydroxide, calcium hydroxide, magnesium hydroxide, calcium carbonate and ammonia.
